(19) United States
(12) Patent Application Publication
(10) Pub. No.: US 2008/0233587 A1
(43) Pub. Date: Sep. 25, 2008

(54) METHOD FOR DIRECT AMPLIFICATION FROM CRUDE NUCLEIC ACID SAMPLES

(75) Inventors: Dennis Y. Wang, Dublin, CA (US); Lori K. Hennessy, San Mateo, CA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 12/054,174

(22) Filed: Mar. 24, 2008

Related U.S. Application Data
(60) Provisional application No. 60/896,668, filed on Mar. 23, 2007.

(51) Int. Cl. C12Q 1/68; C12P 19/34; C07H 21/04

(52) U.S. Cl. 435/6; 435/91.2; 536/23.1

(57) ABSTRACT

The present teachings relate to improved methods, kits, and reaction mixtures for amplifying nucleic acids. In some embodiments a novel direct buffer formulation is provided which allows for the direct amplification of the nucleic acids in a crude sample with minimal sample purification.

### CROSS REFERENCE TO RELATED APPLICATIONS

This application claims priority benefit under 35 U.S.C. § 119(e) from U.S. Application No. 60/896,668 filed Mar. 23, 2007, and U.S. application Ser. No. 11/750,316, filed May 17, 2007, the contents of which are incorporated herein by reference.

### FIELD

The present teachings generally relate to methods of directly amplifying nucleic acids from crude samples.

### INTRODUCTION

Rapid and accurate detection of DNA profiles is a key aspect of forensic casework sample analysis. Crude samples such as blood and buccal swabs contain substances that can inhibit the activity of the polymerases used for PCR-based short tandem repeat (STR) typing. Historically, it has been necessary to remove inhibitors and purify DNA before performing downstream enzymatic manipulations such as PCR amplification. Many kinds of DNA isolation and purification methods and kits are commercially available. However, their use adds time and expense.

### SUMMARY

The present teachings provide a method of performing a polymerase chain reaction (PCR) comprising; providing a crude sample comprising deoxyribonucleic acid;

optionally incubating with NaOH at 5 mM to 25 mM; mixing the crude sample with a direct buffer; and performing a PCR on the deoxyribonucleic acid, wherein the direct buffer comprises at least 5 PCR primer pairs, Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM. and DMSO at 0-4%

In some embodiments, the direct buffer comprises, Tris-HCl at 10 mM pH 8.3, KCl at 50 mM, dNTPs at 200 uM each dNTP, BSA at 800 ug/ml, AmpliTaq Gold polymerase at 0.16 units/ul, MgCl2 at 1.6 mM, DMSO at 2%

In some embodiments, the direct buffer further comprises Sodium Azide at 0.02 percent.

In some embodiments, the present teachings provide a method of determining the identity of a human comprising; providing a crude sample comprising deoxyribonucleic acid from the human; optionally incubating the crude sample with NaOH at 5 mM to 25 mM, mixing the crude sample with the direct buffer, wherein the direct buffer comprises a plurality of primer pairs, wherein each primer pair flanks a genomic locus containing a short tandem repeat (STR); performing a PCR on the deoxyribonucleic acids from the crude sample to form a plurality of PCR amplicons, wherein each PCR amplicon has an ascertainable size; and, identifying the human by reference to size of the PCR amplicons, wherein the direct buffer further comprises Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM, and DMSO 0-4%.

In some embodiments, the present teachings provide a method of preparing nucleic acids for a downstream enzymatic manipulation comprising; providing a crude sample comprising deoxyribonucleic acid; optionally incubating the crude sample with NaOH at 5 mM to 25 mM; mixing the crude sample with a direct buffer; and performing a downstream enzymatic manipulation on the eluate, wherein the direct buffer comprises Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM, and DMSO at 0-4%

In some embodiments, the downstream enzymatic manipulation is a PCR.

In some embodiments, the present teachings provide a kit comprising; a plurality of primer pairs, wherein each primer pair flanks a genomic locus containing a short tandem repeat (STR); and, a direct buffer, wherein the direct buffer comprises Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM, and DMSO at 0-4%.

In some embodiments, the present teachings provide a reaction mixture comprising a direct buffer and a plurality of primer pairs, wherein each primer pair flanks a genomic locus containing a short tandem repeat (STR); and wherein the direct buffer comprises Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM, and DMSO at 0-4%.

In some embodiments the present teaching provide kits as set forth above with optional reagent NaOH or other strong alkaline compounds.

### DESCRIPTION OF EXEMPLARY EMBODIMENTS

In this application, the use of the singular includes the plural unless specifically stated otherwise. In this application, the word “a” or “an” means “at least one” unless specifically stated otherwise. In this application, the use of “or” means “and/or” unless stated otherwise. Furthermore, the use of the term “including,” as well as other forms, such as “includes” and “included,” is not limiting.

The section headings used herein are for organizational purposes only and are not to be construed as limiting the subject matter described. All documents, or portions of documents, cited in this application, including but not limited to patents, patent applications, articles, books, and treatises are hereby expressly incorporated by reference in their entirety for any purpose. In the event that one or more of the incorporated documents defines a term that contradicts that term's definition in this application, this application controls.

### SOME DEFINITIONS

As used herein, the term “crude sample” refers to a specimen of biological origin suspected of containing nucleic acids, which has not undergone substantial procedures for the isolation of those nucleic acids. For example, a sample of blood is a crude sample. Further, a sample of blood spotted on a paper, such as FTA paper commercially available from Whatman, is a crude sample. A buccal swab of cheek cells is another example of a crude sample. Blood, diluted blood, blood on paper, and buccal swabs are illustrative crude samples. One of skill in the art will recognize an enormous variety of other crude samples whose analysis would be facilitated by the present teachings.

As used herein, the term “direct buffer” refers to a buffer into which a crude sample can be placed. The direct buffer contains primers and enzyme for performing a downstream enzymatic manipulation, such as a polymerase chain reaction (PCR). The direct buffer allows for the liberation of the nucleic acids, and for their amplification from the eluate, without the need for any other purification. Illustrative cycling times and temperatures for PCR can be found in Sambrook et al., Molecular Cloning, 3^{rd} Edition 1993. While the present teachings focus on the use of the direct buffer for PCR, it will be appreciated that one of skill in the art can easily employ the direct buffer of the present teachings as a front-end procedure for other types of downstream enzymatic manipulations, for example reverse transcription using a reverse transcriptase, or an oligonucleotide ligation assay using a ligase.

A strong alkaline compound as used herein includes but not limited to NaOH.

### DETAILED DESCRIPTION

A large number of experiments were performed, varying the respective concentration of each of the ingredients of a desired direct buffer, including Tris-HCl, KCl, dNTPs, BSA, AmpliTaq Gold polymerase, MgCl2, and single stranded binding protein (SSB). These experiments used, for example, humic acid as a mimic for the inhibitors typically present in difficult to analyze samples of biological material, and hence served as an easy to produce proxy for crude samples. The results of these experiments yielded the following formulations.

In some embodiments, the present teachings provide a direct buffer comprising Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM, DMSO at 0-4%.

In some embodiments, the direct buffer comprises Tris-HCl at 10 mM pH 8.3, KCl at 50 mM, dNTPs at 200 uM each dNTP, BSA at 800 ug/ml, AmpliTaq Gold polymerase at 0.16 units/ul, MgCl2 at 1.6 mM, and DMSO at 2%.

In some embodiments, the direct buffer further comprises sodium azide, for example at 0.2 percent.

The reagents used in the direct buffer are readily available from commercial suppliers. For example, AmpliTaq Gold is commercially available for Applied Biosystems. BSA is commercially available from a variety of sources, for example catalog number 10711454001 from Roche. FTA paper is commercially available from Whatman.

In some embodiments, FTA paper is used herein with bloodstains. For example, Bloodstain Card from Whatman (Cat# WB 10 0014).

In some embodiments, the FTA paper is punched at 0.5 mm to 1.2 mm. In some embodiments, the punch is 1.5 mm.

In some embodiment, the paper is placed directly to the PCR mix with direct buffer for PCR reaction. In some embodiment, the wash of the paper is not required.

In some embodiments, when non-FTA paper is used, a NaOH solution at 5-25 mM is incubated with non-FTA before PCR reaction.

In some embodiments, the direct buffer comprises a plurality of PCR primer pairs. For example, in some embodiments, the direct buffer comprises 5 primer pairs. In some embodiments, the direct buffer comprises 10 primer pairs. In some embodiments, the direct buffer comprises greater than 10 primer pairs. In some embodiments, the direct buffer does not comprise PCR primer pairs, but rather the PCR primer pairs. are added at a separate time.

### EXAMPLES

In a first example, blood was applied to FTA paper (Whatman) and air-dried. A 0.5 mm disc punch of FTA paper was made and placed into the direct buffer containing PCR primers from the commercially available Identifiler Human Identity Kit (Applied Biosystems). PCR was then performed.

In a second example, 100-fold dilutions of blood were made with TE buffer (10 mM Tris-Cl and 0.1 mM EDTA at pH 8.0). 1 ul of diluted blood was used to set up a PCR in the direct buffer.

In a third example, buccal swab samples were collected and placed in 500 ul TE buffer. The resulting suspension was heated at 97 C for 5 minutes. 10 ul of the resulting suspension was used to set up a PCR in the direct buffer.

In a fourth example, for non-FTA Cards, a 0.5 mm punch is mixed with NaOH at 10 mM and incubate at least 30 seconds or 1 minute, add 15 ul of direct buffer containing PCR primers to start PCR reaction.

### Exemplary Kits in Accordance with Some Embodiments of the Present Teachings

In some embodiments, the present teachings also provide kits designed to expedite performing certain methods. In some embodiments, kits serve to expedite the performance of the methods of interest by assembling two or more components used in carrying out the methods. In some embodiments, kits may contain components in pre-measured unit amounts to minimize the need for measurements by end-users. In some embodiments, kits may include instructions for performing one or more methods of the present teachings. In certain embodiments, the kit components are optimized to operate in conjunction with one another.

While the present teachings have been described in terms of these exemplary embodiments and experimental data, the skilled artisan will readily understand that numerous variations and modifications of these exemplary embodiments are possible without undue experimentation. All such variations and modifications are within the scope of the current teachings.

Thus, in some embodiments, the present teachings provide a kit comprising; a plurality of primer pairs, wherein each primer pair flanks a genomic locus containing a short tandem repeat (STR); and, a direct buffer, wherein the direct buffer comprises Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM, and DMSO at 0-4% and optionally sodium azide at 0.02%, and optionally NaOH at 5 mM to 25 mM. Such a kit can be used, for example, in the identification of an organism such as a human by the collection of polymorphic microsatellites analyzed, using for example capillary electrophoresis. Illustrative procedures for performing such human identification can be found for example in the Identifiler HID kit, commercially available from Applied Biosystems, as well as U.S. Pat. Nos. 6,221,598, 6,479,235, 5,843,660, and 7008771. In some embodiments, the kits, and methods and reaction mixtures provided by the present teachings can be used with procedures for multiplexed PCR of degraded samples, as found for example in WO05054515 to Dimsoski and Woo.

In some embodiments, the direct buffer in the kit comprises, Tris-HCl at 10 mM pH 8.3, KCl at 50 mM, dNTPs at 200 uM each dNTP, BSA at 800 ug/ml, AmpliTaq Gold polymerase at 0.16 units/ul, MgCl2 at 1.6 mM, and DMSO at 0-4%.

In some embodiments, the direct buffer in the kit further comprises Sodium Azide at 0.02 percent.

In some embodiments, the direct buffer in the kit future comprises NaOH at 5 mM-25 mM.

All literature and similar materials cited in this application, including but not limited to, patents, patent applications, articles, books, treatises, and internet web pages, regardless of the format of such literature and similar materials, are expressly incorporated by reference in their entirety for any purpose.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

1. A method of performing a polymerase chain reaction (PCR) comprising;
providing a crude sample comprising deoxyribonucleic acid;
optionally incubating with NaOH,
mixing crude sample with a direct buffer; and
performing a PCR on the deoxyribonucleic acid, wherein the direct buffer comprises at least 5 PCR primer pairs, Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM, and DMSO at 0-4%.

2. The method according to claim 1 wherein the direct buffer comprises, Tris-HCl at 10 mM pH 8.3, KCl at 50 mM, dNTPs at 200 uM each dNTP, BSA at 800 ug/ml, AmpliTaq Gold polymerase at 0.16 units/ul, MgCl2 at 1.6 mM, DMSO at 2%

3. The method according to claim 2 wherein the direct buffer further comprises Sodium Azide at 0.02 percent.

4. A method of determining the identity of a human comprising;
providing a crude sample comprising deoxyribonucleic acid from the human;
optionally incubating with NaOH;
mixing the crude sample with a direct buffer, wherein the direct buffer comprises a plurality of primer pairs, wherein each primer pair flanks a genomic locus containing a short tandem repeat (STR);
performing a PCR on the deoxyribonucleic acids from the crude sample to form a plurality of PCR amplicons, wherein each PCR amplicon has an ascertainable size; and, identifying the human by reference to size of the PCR amplicons, wherein the direct buffer further comprises Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM, and DMSO at 0-4%.

5. The method according to claim 4 wherein the direct buffer comprises, Tris-HCl at 10 mM pH 8.3, KCl at 50 mM, dNTPs at 200 uM each dNTP, BSA at 800 ug/ml, AmpliTaq Gold polymerase at 0.16 units/ul, MgCl2 at 1.6 mM, and DMSO at 0-4%.

6. The method according to claim 5 wherein the direct buffer further comprises Sodium Azide at 0.02 percent.

7. A method of preparing nucleic acids for a downstream enzymatic manipulation comprising;
providing a crude sample comprising deoxyribonucleic acid;
optionally incubating crude sample with NaOH;
mixing the crude sample with a direct buffer; and
performing a downstream enzymatic manipulation on the mixture, wherein the direct buffer comprises Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM, and DMSO at 0-4%.

8. The method according to claim 7 wherein the downstream enzymatic manipulation is a PCR.

9. The method according to claim 7 wherein the direct buffer comprises, Tris-HCl at 10 mM pH 8.3, KCl at 50 mM, dNTPs at 200 uM each dNTP, BSA at 800 ug/ml, AmpliTaq Gold polymerase at 0.16 units/ul, MgCl2 at 1.6 mM, and DMSO at 2%.

10. The method according to claim 9 wherein the direct buffer further comprises Sodium Azide at 0.2 percent.

11. A kit comprising;
a plurality of primer pairs, wherein each primer pair flanks a genomic locus containing a short tandem repeat (STR); and,
a direct buffer, wherein the direct buffer comprises Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM, and DMSO at 0-4%.

12. The kit according to claim 11 wherein the direct buffer comprises, Tris-HCl at 10 mM pH 8.3, KCl at 50 mM, dNTPs at 200 uM each dNTP, BSA at 800 ug/ml, AmpliTaq Gold polymerase at 0.16 units/ul, MgCl2 at 1.6 mM, and DMSO at 0-4%.

13. The kit according to claim 12 wherein the direct buffer further comprises Sodium Azide at 0.02 percent.

14. A reaction mixture comprising a direct buffer and a plurality of primer pairs, wherein each primer pair flanks a genomic locus containing a short tandem repeat (STR); and wherein the direct buffer comprises Tris-HCl at 10-16 mM, KCl at 25-75 mM, dNTPs at 200-400 uM each dNTP, BSA at 160-960 ug/ml, AmpliTaq Gold polymerase at 2 U-8 U, MgCl2 at 1.25-2.2 mM, and DMSO at 0-4%.

15. The reaction mixture according to claim 14 wherein the direct buffer comprises, Tris-HCl at 10 mM pH 8.3, KCl at 50 mM, dNTPs at 200 uM each dNTP, BSA at 800 ug/ml, AmpliTaq Gold polymerase at 0.16 units/ul, MgCl2 at 1.6 mM, and DMSO at 0-4%.

16. The reaction according to claim 15 wherein the direct buffer further comprises Sodium Azide at 0.02 percent.
